(19) United States
(12) Patent Application Publication
(10) Pub. No.: US 2026/0092306 A1
(43) Pub. Date: Apr. 2, 2026

(54) DETECTION OF DIAGNOSTIC BIOMARKERS IN BODY FLUID SAMPLES

(71) Applicant: DROPLET BIOSCIENCES, INC., Cambridge, MA (US)

(72) Inventors: Wendy Winckler Adamovich, Melrose, MA (US); Seka Lazare, Melrose, MA (US)

(21) Appl. No.: 19/342,251

(22) Filed: Sep. 26, 2025

Related U.S. Application Data
(60) Provisional application No. 63/700,190, filed on Sep. 27, 2024.

(51) Int. Cl. C12Q 1/6806; C12Q 1/37; C12Q 1/6869; C40B 40/06; C40B 50/06; G16B 30/10

(52) U.S. Cl. CPC C12Q 1/6806; C12Q 1/37; C12Q 1/6869; C12Y 304/21064; C40B 40/06; C40B 50/06; G16B 30/10; C12Q 2600/112; C12Q 2600/118

(57) ABSTRACT

The invention provides methods for cancer diagnosis, for predicting recurrence and minimal residual disease, and for informing therapeutic choice/predicting therapeutic efficacy. The invention comprises extracting cfDNA from a bodily fluid sample and performing size selection to extract cfDNA from the sample. The size selection step may be performed any number of times as is suitable to obtain a desired yield.

### BACKGROUND

Disease diagnostics and recurrence monitoring are important components in the prevention and management of diseases with a genomic basis, such as cancer. For example, in cancer, recurrence is a significant cause of mortality, with up to 50% of patients recurring within 2 years. Moreover, no reliable methods exist to tailor adjuvant therapy for individual patients based on risk of recurrence after surgery. While treatment selection considers pathological risk factors, such as HPV status, tumor stage or extranodal extension (ENE), 5-year overall survival rates remain low. Treatment approaches, such as aggressive adjuvant radiotherapy (RT) or chemotherapy plus radiotherapy (CRT), have been associated with improvements in both progression free survival (PFS) and overall survival in cancer patients, but often result in high morbidity, as well as physiological, financial and emotional burden.

Thus, more accurate diagnostics in body fluid samples and identification of patients who would most benefit from escalated treatment remain critical needs. For example, detection of HPV viral DNA is prognostic in HPV-positive head and neck cancer. However, plasma cfDNA has not been prognostic and has generally failed to detect recurrence. Moreover, cfDNA is generally not first detected until months after surgery. In addition, cfDNA is difficult to detect in a sample that is compromised by an increased presence of genomic DNA due to biological mechanisms or technical mishandling of a biological sample. Accordingly, there is a need for methods of detecting cfDNA early in disease prognosis in body fluid samples.

### SUMMARY OF THE INVENTION

The invention provides methods for cancer diagnosis, for predicting recurrence and minimal residual disease, and for informing therapeutic choice/predicting therapeutic efficacy. The invention provides methods for detecting diagnostic biomarkers, especially cfDNA, in compromised body fluid samples in which the presence of genomic DNA confounds detection of cfDNA in the sample. Accordingly, the invention allows early characterization and prognosis of disease that is critical in assessing patient status and a course of treatment.

Pre-analytical variables, such as sample shipping and handling, have been shown to affect quality of isolated cfDNA. Genomic DNA contamination from lysing of cells during handling or transport can confound the detection of rare variants, like those in most liquid biopsy applications. Methods of the invention reduce genomic DNA contamination from compromised blood, serum or other biofluids, thereby enriching the cell-free DNA fraction for downstream analysis. Methods of the invention are especially applicable to compromised blood, serum or other biofluids, or cell-free DNA that has been isolated and has shown a high percentage of genomic DNA contamination (e.g. >20%, over 30% etc. DNA >600 bp). In one aspect, the invention comprises extracting DNA from these sources, removing genomic DNA by selectively binding large DNA molecules (e.g. >600 bp) and subsequently binding and purifying the enriched cell-free nucleic acid fraction. In one example, beads, such as magnetic or paramagnetic beads, are used to remove the gDNA. Where the method is applied to compromised blood, serum or other biofluids, the samples may first be treated with enzymes or proteins to aid in cell-free DNA release and degrade proteins that may inhibit downstream processes, e.g. proteinase k, prior to genomic DNA removal and cell-free nucleic acid purification.

In particular aspects, methods of the invention provide the ability to detect cfDNA in a sample in which detection of cfDNA would be difficult or impossible to detect using conventional techniques. Extraction of cfDNA from various body fluid sources in which the cfDNA is reduced in content relative to genomic DNA allows increased sensitivity and specificity of detection, regardless of the sample source. Methods of the invention are useful in samples that are compromised by having increased genomic DNA content as a result of biological processes and/or technical handling of the sample. Preferred samples include samples in which a biological process or sample handling have resulted in a comprised body fluid sample, e.g., a sample in which the amount of gDNA makes extraction of cfDNA difficult due primarily to their relative abundances in the compromised sample. Blood plasma is a preferred sample for use of methods of the invention.

According to the invention, cfDNA is extracted from a body fluid sample using smaller input volumes and at significantly higher yields than would be possible in conventional isolation from plasma. In a preferred instance, the invention comprises extracting cfDNA from a bodily fluid sample and performing size selection to extract cfDNA from the sample. The size selection step may be performed any number of times as is suitable to obtain a desired yield. Use of methods disclosed and claimed herein results in a greater yield of extracted cfDNA than would be possible if conventional plasma-based assays are used on the fluid, especially in the case of a compromised fluid. In addition to the extraction of cfDNA, methods of the invention can be applied to the extraction of other analytes, such as RNA and proteins. In preferred methods, extracted cfDNA is sequenced and disease prognosis is predicted based on the sequence analysis of the cfDNA. Prediction of minimal residual disease, for example, is based on comparing cfDNA sequence obtained from the sample to known sequences associated with disease outcome and/or recurrence. The known sequences may be obtained from a database of sequences, may be tumor-informed, or may be otherwise obtained empirically.

In one aspect, methods of the invention comprise extracting nucleic acid from a bodily fluid, size selecting for cell-free nucleic acid, and detecting and/or identifying the size-selected nucleic acid. Certain embodiments of the invention comprise removing genomic DNA (gDNA) from the sample prior to, or simultaneous with, the size selecting step. In one example, beads, such as magnetic or paramagnetic beads, are used to remove the gDNA. In general, the invention is based on the recognition that cfDNA indicative of minimal residual disease (MRD) in body fluid represents an early indication of MRD and recurrence. Methods of the invention are also applicable to non-nucleic acid markers present in lymphatic exudate and that are not present in plasma at similar timepoints. Examples of non-nucleic acid markers include proteins and peptides, carbohydrates, and metalloproteins.

In specific instances, the invention comprises extracting nucleic acid from a body fluid, size selecting for cell-free nucleic acid, and detecting and/or identifying the size-selected nucleic acid. Certain embodiments of the invention comprise removing genomic DNA (gDNA) from the sample prior to, or simultaneous with, the size selecting step. In one example, beads, such as magnetic or paramagnetic beads, are used to remove the gDNA. Thus, the size selection can be in favor of the cfDNA or the gDNA as desired.

Methods of the invention are useful for isolating nucleic acid for diagnosis, prognosis, and assessment of disease (e.g., staging), as well as evaluating therapeutic selection and efficacy. The nucleic acids obtained via methods of the invention can be evaluated by sequencing methods and compared to wild-type or disease model sequences. In addition, a genomic profile created from nucleic acid obtained in the invention is useful to model disease progression. Libraries of, for example, cfDNA made using methods of the invention are useful for the evaluation of samples as well as for creation of a database for analysis of subsequent samples.

In preferred embodiments, the cfDNA is circulating tumor DNA (ctDNA). The ctDNA obtained as described herein can be sequenced and compared to profiles obtained from tumor biopsy samples, blood samples, and/or lymphatic samples. Thus, methods of the invention are useful to create multi-modal genomic profiles that are used to track cancer progress, to detect minimal residual disease (MRD), and to predict recurrence and metastasis. Methods of the invention also may be used to yield quantitative information concerning cfDNA by, for example, amplifying extracted cfDNA using quantitative methods, such as qPCR.

Accordingly, the present invention provides a method for disease diagnosis comprising the steps of obtaining a drain fluid sample, extracting nucleic acid from the sample, conducting a size selection procedure to isolate cell-free nucleic acid in the sample, and detecting the cell-free nucleic acid. In certain embodiments, methods of the invention further include sequencing the cell-free nucleic acid and (i) diagnosing and/or staging cancer and/or (ii) selecting or evaluating a therapeutic. In certain aspects, the cell-free nucleic acid is isolated without a prior step of extracting nucleic acid from the sample. The disease evaluated can be any disease, including cancer, infectious disease, metabolic disease or an autoimmune disease. However, the invention has particular application in the diagnosis, prognosis, and monitoring of cancer.

In a certain embodiment, the invention includes repeating the extracting step at least once. In another embodiment, methods include repeating the conducting step at least once. The cfDNA percentage and/or quantity may be assessed after each extraction or size selection round, and the extraction and/or size selection steps are repeated until the desired percentage and/or quantity of cfDNA is achieved. The desired percentage and/or quantity of cfDNA may correspond to a target detection threshold. In certain embodiments, the desired percentage of cfDNA is at least about 70%. In certain embodiments, the desired quantity of cfDNA is at least about 600 ng. The size selection rounds may comprise the addition of beads. In addition, the invention may further comprise treatment with Proteinase K prior to the addition of beads.

Other aspects and advantages of the invention are apparent to the skilled artisan based on the following claims and detailed description thereof.

### DETAILED DESCRIPTION

The present disclosure provides methods for early identification of indicators of disease, presence or potential for MRD or recurrence, and therapeutic selection and/or efficacy. Methods of the invention are directed to isolation of cell-free nucleic acid in a sample in which large quantities of genomic DNA make extraction of the cell-free fraction difficult. An example of such a sample is one in which a biological process or handling/processing of the sample result in the sample being compromised in the sense that the gDNA fraction makes identification of cfDNA more difficult. According to the invention, a size selection is performed on a body fluid sample to reduce the amount of large genomic DNA fragments that, if left in the sample, would make it difficult to isolate cell-free nucleic acid, which is smaller than genomic fragments and present in lower relative abundance in the sample. Methods of the invention are especially useful in body fluid samples that undergo a biological process or through processing of the sample produce high amounts of genomic DNA. Using size selection according to the invention, large fragments and small fragments are isolated, making it easier to capture the targeted small, cell-free fragments. Methods of the invention are applicable to any bodily fluid, including but not limited to blood (plasma), lymphatic fluid, seroma fluid, cerebral spinal fluid, saliva, and urine.

Preferred methods of the invention comprise extracting cfDNA from a body fluid sample by size selection to remove large genomic fragments and optionally proteins from the sample. In a preferred embodiment, beads, preferably magnetic beads, are used for size selection as described here.

Plasma that has been subjected to biological disruption or improper handling may have high genomic content. For example, some workflows subject a blood sample from a subject to a plasma separation to separate plasma from blood cells. Conventional plasma separation protocols involve centrifugation after which the supernatant representing the plasma component of the sample is retained. It may happen that the plasma separation protocol is performed poorly or in a manner that compromises the resulting plasma sample (supernatant). That is, the protocol may damage or lyse a portion of the cellular component or otherwise incompletely separate the plasma from the whole cells in a manner that leads to unexpected amounts of genomic DNA (gDNA) in the plasma component. Such a plasma sample, after a plasma separation protocol conducted in a manner that compromised the plasma sample with genomic DNA and/or other material such as protein is well-suited to the size-selection-based methods (with optional proteinase treatment) described herein. Such methods reduce or minimize the gDNA contamination in a compromised body fluid sample such as a plasma sample.

Methods to minimize, for example, gDNA contamination include bead purification to size select the DNA fragments in a cfDNA sample. In some embodiments, isolating cfDNA is done in such a manner as to maximize the recovery of short fragments (<100 base pairs), as the composition of short fragments differs more between healthy and disease states than the composition of longer fragments. In some embodiments, the cfDNA fragments are subjected to a size selection to retain only cfDNA fragments having a length between an upper bound and a lower bound. In some embodiments, the upper bound is about 200, about 190, about 180, about 170, about 160, about 150, about 140, about 130, about 120, about 110, about 100, about 90, about 80, about 70, about 60, or about 50 base pairs and the lower bound is about 20, about 25, about 30, about 35, about 36, about 40, about 45, about 50, about 60, about 70, about 80, about 90, about 100, about 110, or about 120 base pairs. In some embodiments, the lower bound is 36 and the upper bound is 100. In some embodiments, the beads are magnetic beads. In some embodiments, the ratio of beads to the drain fluid can be 0.1×, 0.2×, 0.3×, 0.4×, 0.5×, 0.6×, 0.7×, 0.8×, 0.9×, and 1×. In some embodiments, the ratio of magnetic beads may be added in staggered steps, e.g., where 0.4× beads are added, and then separated and an additional 0.1× beads is added to the supernatant.

In various aspects, cfDNA may be identified and quantified using methods known in the art. Suitable assays include, for example, nucleic acid sequencing, PCR, quantitative PCR, digital and droplet PCR. Sequencing may be performed by various methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Tumor-informed plasma-based cfDNA assays for detection of minimal residual disease (MRD) after cancer treatment are limited by 1) sampling bias from a small sampling of the tumor, and 2) tumor heterogeneity and subclonality that develops during the course of carcinogenesis and metastasis. As discussed above, according to the invention, surgical drain fluid in patients undergoing cancer surgery is a good source of tumor cfDNA. The characterization and/or quantification of tumor cfDNA in the surgical drain fluid is useful to measure locoregional minimal residual disease as well as for determining the risk of recurrence. Additionally, cfDNA in drain fluid broadly captures tumor heterogeneity in a manner that is difficult or impossible with the small biopsy sample typically obtained from primary tumor tissue. Thus, drain fluid allows detection of a broader spectrum of variants, including high-risk variants, that represent the entirety of the tumor and lymph nodes metastases. Methods described herein increase the yield of informative cfDNA from tumor sources.

In yet another aspect, the invention provides methods for determining MRD. Tumor DNA extracted using methods of the invention is sequenced and informative or potentially-informative variants are determined. The cfDNA is used to design a customized MRD panel that is then used to monitor the patient for recurrence, disease progression, response to treatment and other clinical signs over a period of time at the discretion of the clinician. An advantage of this aspect of the invention is that a panel constructed, at least in part, from cfDNA variants obtained in body fluid allows for personalized surveillance monitoring of a patient.

1. A method for detecting cell-free nucleic acid in a body fluid sample, the method comprising the steps of
extracting nucleic acid from a body fluid sample;
conducting a size selection procedure to isolate cell-free nucleic acid from the sample; and
detecting the cell-free nucleic acid.

2. The method of claim 1, wherein the cell-free nucleic acid is DNA.

3. The method of claim 1, wherein the extracting step is repeated at least once.

4. The method of claim 1, wherein the body fluid sample is plasma.

5. The method of claim 4, wherein the plasma sample has been compromised.

6. The method of claim 1, further comprising the step of removing genomic DNA prior to the conducting step.

7. The method of claim 6, wherein the removing step comprises capturing genomic DNA on beads.

8. The method of claim 7, wherein the beads are magnetic beads.

9. The method of claim 1, further comprising sequencing the cell-free nucleic acid.

10-11. (canceled)

12. The method of claim 1, further comprising the step of preparing a genomic profile from nucleic acid in the sample.

13. The method of claim 1, further comprising the step of identifying minimal residual disease in a patient from whom the sample was obtained.

14. A method for cancer diagnosis, the method comprising the steps of:
conducting a size selection procedure to isolate cell-free nucleic acid in body fluid sample; and
detecting the cell-free nucleic acid.

15. The method of claim 14, wherein the size selection procedure comprises the addition of beads to the sample.

16. The method of claim 15, further comprising the step of treating the sample with proteinase K prior addition of the beads.

17-18. (canceled)

19. A method for stratifying risk of recurrence in cancer patients, the method comprising the steps of:
obtaining a lymphatic exudate sample from a patient;
analyzing the sample to identify cfDNA that is detectable in the sample but not in a contemporaneously-obtained blood sample;
determining a sequence of the cfDNA; and
stratifying risk of cancer recurrence by analyzing the sequence of the cfDNA.

20. The method of claim 19, wherein the stratifying step further comprises comparing the cfDNA to a database of associates with increased risk of recurrence.

21. The method of claim 19, wherein the stratifying step is based on an amount of cfDNA identified in the sample.

22-28. (canceled)

29. The method of claim 19, wherein the lymphatic exudate is obtained via an implanted drain.

30. The method of claim 19, wherein the cfDNA is not found in a contemporaneously obtained blood sample.

31. The method of claim 19, wherein the cfDNA is first detectable in blood about two weeks subsequent to being detectable in the lymphatic exudate.
